(19) United States
(12) Patent Application Publication
(10) Pub. No.: US 2017/0215796 A1
(43) Pub. Date: Aug. 3, 2017

(54) SKIN MEASURING DEVICE AND WRISTWATCH

(71) Applicant: PYREOS LTD., Edinburgh (GB)

(72) Inventors: Carsten Giebeler, Edinburgh (GB); Hugo Vargas Llanas, Edinburgh (GB)

(21) Appl. No.: 15/494,266

(22) Filed: Apr. 21, 2017

Related U.S. Application Data
(63) Continuation of application No. PCT/EP2015/074613, filed on Oct. 23, 2015.

(30) Foreign Application Priority Data
Oct. 24, 2014 (DE) 10 2014 115 502.5

(51) Int. Cl. A61B 5/00

(52) U.S. Cl. CPC A61B 5/681; A61B 5/0015; A61B 5/0075; A61B 5/443

(57) ABSTRACT

A skin measuring device for spectroscopic measurement of the skin of a body part is provided. The skin measuring device has a press-on frame with a window and a flat face. The skin measuring device has an ATR infrared spectrometer which includes an ATR crystal that is secured to the press-on frame and that has a sample stage which is arranged in the window and faces in the same direction as the flat face of the press-on frame. An encircling means surrounds the body part and thereby supports the skin measuring device on the body part. The surface of the flat face of the press-on frame is pressed against the skin of the body pan by the encircling means in a comfortably wearable manner when the skin measuring device is worn such that the sample stage is in contact with the skin for spectroscopic measurement by the ATR spectrometer.

### CROSS REFERENCE TO RELATED APPLICATIONS

This application is a continuation application of international patent application PCT/EP2015/074613, tiled Oct. 23, 2015, designating the United States and claiming priority from German application 10 2014 115 502.5, filed Oct. 24, 2014, and the entire content of both applications is incorporated herein by reference.

### TECHNICAL FIELD

The invention relates to a skin measuring device and a wristwatch that holds the skin measuring device.

### BACKGROUND

Many people have a desire to improve themselves physically, for example by reducing their body weight or increasing their stamina. In order to monitor physical progress, portable devices are available, which, record data of the body of the exercising person and provide it to the exercising person during training. With these devices, there is a need for further miniaturization to achieve a high wearing comfort. Furthermore, it is desirable that diversified data records of the body be recorded, such as, parameters of the exerciser's skin climate during training, which, in particular, allow conclusions to be drawn about the quality of the metabolism of the exercising person during training.

### SUMMARY

It is an object of the invention to provide a skin measuring device and a wristwatch that holds the skin measuring device, whereby the skin measuring device is configured to measure diversified data of the skin and yet has a high wearing comfort with small dimensions.

The skin measuring device for the spectroscopic measurement of a skin of a body part according to an aspect of the invention has a press-on frame with a window, and an ATR infrared spectrometer having an ATR crystal attached to the press-on frame. A sample stage is arranged in the window and thus engages with the press-on frame and faces in the same direction as one flat face of the press-on frame. The device further has an encircling means that is configured to surround the body part and thereby to support the skin measuring device on the body part. The press-on frame is pressed onto the skin of the body part by the encircling means when the skin measuring device is being worn, such that the sample stage is in contact with the skin and thereby the spectroscopic measurement of the skin can be accomplished with the. ATR infrared spectrometer.

In the ATR infrared spectrometer, infrared radiation interacts with the skin that is in contact with the sample stage. Evanescent waves are formed in the skin, which have a range of approximately one wavelength from the sample stage. Surprisingly, it has been found that when the press-on frame is pressed with an encircling means that does neither exert too much pressure that would be felt as being unpleasant, nor causes too little pressure which would be felt to be too loose, an advantageous contacting of the ATR crystal with the skin occurs. Thus, evanescent waves are formed in the skin and result in a spectroscopic measurement of the skin that is particularly precise. Thus, the skin measuring device is at the same time easy to carry and allows precise spectroscopic measurements of the skin.

In contrast to conventional absorption infrared spectrometers, the ATR infrared spectrometer can be made substantially smaller and has a substantially lower energy consumption. This makes it particularly suitable for being worn as a mobile device on the body. The ATR infrared spectrometer is used to determine a variety of information from the skin. For example, the spectrometer can determine the water content of the skin.

According to an aspect of the invention, the body part is an arm of a human being and the encircling means is a wristband. An especially high wearing comfort of the skin measuring device can advantageously be achieved on the arm. The wristband is preferably elastic and its length is adapted to the body part such that the wristband is elongated when the skin measuring device is worn, and the press-on frame with its one flat face is pressed against the skin of the body part. With the elastic wristband, the press-on frame can be pushed in a particularly simple manner while simultaneously achieving a comfortable contacting of the skin with the sample stage. In addition, for a given length of the wristband, comfortable pressing can be achieved with simultaneously high-quality contacting of the skin liar arms that have different thicknesses.

According to another aspect of the invention, the ATR crystal is arranged in the window in such a way that the sample stage and the fiat face of the press-on frame lie in one and the same plane. Thus, no edges are formed by the press-on frame and the ATR crystal, such that the wearing comfort of the skin measuring device is particularly high. At the same time, optimum contact between the sample stage and the skin is ensured. In addition, the ATR crystal is pressed flatly against the skin by the two-dimensional pressing of the press-on frame onto the skin. As a result, mechanical stresses in the ATR crystal and thus also a stress birefringence of the ATR crystal is prevented. The stress birefringence of the crystal would cause a falsification of the infrared spectra measured with the skin measuring device. By arranging the sample stage and the two-dimensional side of the press-on frame in the same plane, the infrared spectra of the skin can thus be measured without stress birefringence and with high accuracy.

According to yet another aspect of the invention, the skin measuring device has a housing with a housing base, which is arranged in the press-on frame. This accommodates the ATR infrared spectrometer in the housing, thus shielding it from external influences, for example, from external light. The spectroscopic measurements can be carried out with a high degree of accuracy by the shielding.

According to a further aspect of the invention, the material of the ATR crystal has a refractive index n₁ of the ATR crystal that is greater than 1.5 in the range of the infrared radiation. This advantageously ensures that, if the refractive index of the skin is assumed to lie at 1.3, even if the optical properties of the skin fluctuate, which would lead to an increase in the refractive index normally of below 0.2, a total reflection within the ATR crystal occurs.

According to an aspect of the invention, the material of the ATR crystal has a refractive index between 2.3 to 2.8 in the infrared radiation region. In this region, a still sufficient penetration depth of the evanescent waves into the skin is achieved, while at the same time the limiting angle of the total reflection is so small that a sufficiently large number of total reflections can occur in the ATR crystal. In addition, a high number of infrared-transmissive materials are available for the ATR crystal in this range of the refractive index. According to another aspect of the invention, the ATR crystal has two reflection faces that are inclined towards the sample stage. The inclination angle of the reflection faces is selected in such a way that the infrared light beam strikes the sample stage at an angle θ, which is in the range of arcsine (1.3/n₁)+5° to arcsine (1.3/n₁)+10°, where θ is the angle of the infrared radiation enclosed with a line that is perpendicular to a plane of the sample stage. As a result, total reflection can also occur when the optical properties of the skin vary and, at the same time, a large penetration depth for the evanescent waves into the skin is ensured.

According to an aspect of the invention, the skin measuring device has a device connector through which the spectroscopic data that is gathered by the skin measuring device can be read out and/or a transmitting unit through which the spectroscopic data gathered by the skin measuring device can be transmitted wirelessly. The skin measuring device preferably has an analysis unit which is configured to read out the spectroscopic data via the device connector and/or to receive the data transmitted by the transmitter unit wirelessly, and which is configured to evaluate the spectroscopic data.

The wristwatch according to another aspect of the invention contains the skin measuring device. Preferably, the wristwatch has a dial which is arranged facing away from the sample stage. The spectroscopic measurement of the skin and a time measurement are thereby advantageously made possible at the same time.

### BRIEF DESCRIPTION OF THE DRAWINGS

The invention will now be described with reference to the drawings wherein:

FIG. 1 shows a perspective view of a skin measuring device according to an exemplary embodiment of the invention.

FIG. 2 is a plan view of an ATR infrared spectrometer.

FIG. 3 is a side view of the. ATR infrared spectrometer of FIG. 2.

FIG. 4 shows a perspective view of a wristwatch with a view of its rear side, and

FIG. 5 shows a perspective view of the wristwatch from FIG. 4 with a view of its front side.

### DESCRIPTION OF EXEMPLARY EMBODIMENTS

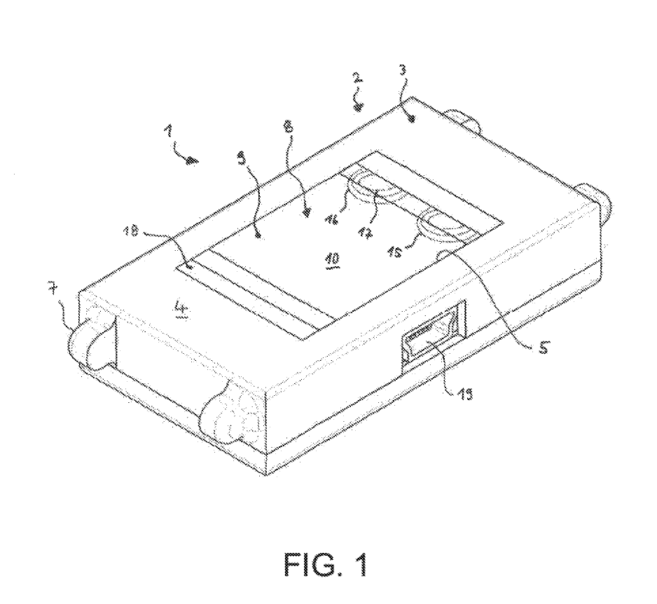

FIG. 1 shows a skin measuring device 1, which has a housing 2 and an ATR infrared spectrometer 8. The ATR infrared spectrometer 8 is arranged inside the housing 2. The housing 2 has a press-on frame 3 in which a window 5 in the form of a rectangular opening is arranged. The housing 2 also has a housing base 4, which is the press-on frame 3.

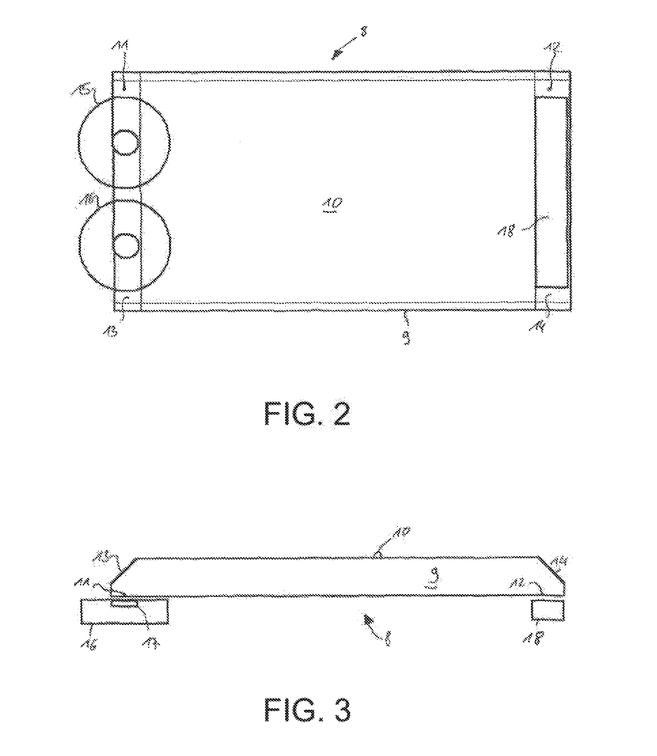

As can be seen in FIGS. 2 and 3, the ATR (“attenuated total reflection”) infrared spectrometer 8 has an ATR crystal 9. The ATR crystal 9 is elongated so that it has a longitudinal axis and two opposing longitudinal faces arranged parallel to the longitudinal axis. One of the two longitudinal faces forms a sample stage 10, which is to be placed in contact with the skin when the skin measuring device 1 is used. The dimensions of the sample stage 10 are essentially the same as the dimensions of window 5. The ATR infrared spectrometer 8 further includes a first inflated light source 15 and a second infrared light source 16, as well as a infrared light line detector 18, which is arranged on the longitudinal side of the ATR crystal 9 that is opposite to the sample stage 10. However, any number of infrared light sources can be used. The infrared light detector line 18 may have any number of infrared light detectors. The first infrared light source 15 and the second infrared light source 16 each have an exit opening 17 through which the light generated by the infrared light sources 15, 16 can be coupled into the ATR crystal 9.

The ATR crystal 9 has a first reflection face 13 at a first longitudinal end and a second reflection face 14 at a second longitudinal end, which is arranged opposite the first longitudinal end. The reflection faces 13, 14 are inclined relative to the longitudinal sides. As a result, the infrared light that is coupled into the ATR crystal 9 in the region of the first longitudinal end via an entrance face 11, which is arranged on the longitudinal side opposite to the sample stage 10, is interreflected between the two longitudinal sides and arrives at the second longitudinal end where the infrared light is decoupled from the ATR crystal 9 via an exit face 12, which is arranged in the longitudinal side opposite the sample stage 10.

After exiting the ATR crystal 9, the infrared light strikes an infrared light line detector 18. The infrared light line detector 18 is formed by a plurality of infrared light detectors arranged side by side in a direction perpendicular to the longitudinal axis of the ATR crystal 9. The infrared light detectors can, for example, be pyroelectric detectors, each having a thin layer of lead zirconate titanate. In order to allow a spectrally resolved measurement, a dispersion medium is arranged between the exit face 12 and the infrared light line detector 18. The dispersant medium is, for example, a wavelength filter, whereby the wavelength range in which the wavelength filter allows the infrared light to pass through varies linearly in the direction perpendicular to the longitudinal axis of the ATR crystal 9.

When using the skin measuring device 1, the sample stage 10 is to be brought into contact with the skin, so that a spectroscopic measurement of the skin can take place. For this purpose, it is necessary for the refractive index of the ATR crystal 9 to be greater than the refractive index of the skin so that a total reflection of the infrared light can take place on the sample stage 10. Because skin mainly consists of water, it is advantageous that the refractive index of the ATR crystal in the range of the infrared light is greater than 1.5. In the skin, by way of total reflection, evanescent waves are formed, which have a range in the order of the wavelength of the infrared light from the sample stage 10 and which interact with the skin. From the spectrum of the infrared light detected by the infrared light detector line 18 spectroscopic properties of the skin can be inferred because of this interaction. A refractive index of 2.3 to 2.8 is advantageous to achieve a long penetration depth of the evanescent waves into the skin and a sufficiently small limiting angle for the total reflection in order to achieve a sufficiently high number of total reflections within the ATR crystal 9. Suitable materials for the ATR crystal 9 are in particular ZnSe, thallium bromide iodide, diamond or AMTIR (“amorphous material transmitting infrared material”), such as Ge₃₃As₁₂Se₅₅, AsₓSeₓ, GeₓSbₓSeₓ or As₂S₃.

FIG. 1 shows that the ATR crystal 9 engages with the press-on frame 3 in such a way that the sample stage 10 is arranged in the window 5 and is directed towards the outside of the housing 2 so that the sample stage is connected to the skin when the skin measuring device 1 is used. The infrared light sources 15, 16 and the infrared light detector line 18, on the other hand, are arranged inside the housing 2. The sample stage 10 lies entirely in the same plane as the surface of the press-on frame 3 that is directed towards the outside of the housing 2. The sample stage 10 and the surface of the press-on frame 3 of the housing 2 that is directed outwardly together form a continuous surface of the skin measuring device 1, which can be attached to the skin in a comfortably wearable manner. The surface of the press-on frame 3 directed towards the outside of the housing 2 is a smooth surface without projections projecting from the surface.

In order to avoid the formation of mechanical stresses in the ATR crystal 9, a resilient O-ring can be provided, by which the ATR crystal is brought into engagement with the press-on frame 3. For this purpose, a circumferential groove into which the O-ring is inserted can be provided in the press-on frame 3. By pressing the ATR crystal 9 with a holding deice against the O-ring, the ATR crystal 9 can be engaged in a stress free manner with the press-on frame 3 while the housing 2 can be sealed with the O-ring.

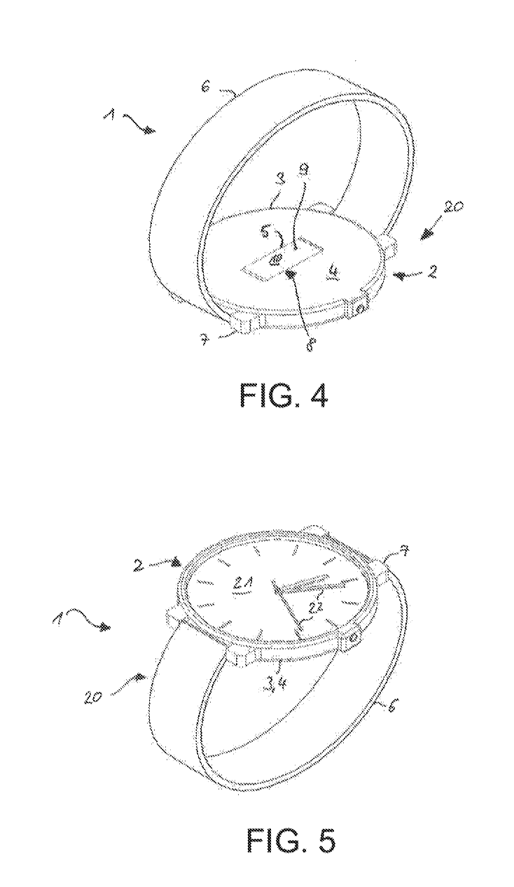

FIGS. 4 and 5 show that the skin measuring device 1 further includes an encircling means 6 which is configured in such a way that a body part can be surrounded by the encircling means 6 and thus the skin measuring device 1 can be worn on the body part. The skin measuring device 1 can be worn by the encircling means 6 in such a manner that it neither causes excessive pressure on the body part, nor is carried around the body part such that it can move uncontrollably on the body part. As a result, at the same time, the housing base 4 and the sample stage 10 are pressed flatly against the skin of the body part, and the spectroscopic, measurement of the skin can be accomplished.

As shown in FIGS. 1, 4, and 5, the skin measuring device 1 on the housing 2 has two pairs of lugs 7, which are mounted on opposite side faces of the housing 2 and which extend substantially perpendicular to the housing base 4. In order to fasten the encircling means 6 to the housing 2, a longitudinal end of the encircling means 6 engages in each case with one of the pairs of the lugs.

The encircling means 6 shown in FIGS. 4 and 5 is a wristband or strap configured to encircle an arm of a human being. It is conceivable to design the wristband elastically and to adapt its length to the thickness of the arm, such that the wristband is elongated when the skin measuring device 1 is worn on the arm and the housing base 4 together with the sample stage 10 is flat and comfortably pressed on the skin of the arm. At the same time, the sample stage is in contact with the skin of the arm in such a way that a spectroscopic measurement of the skin can be accomplished with the ATR infrared spectrometer.

In FIGS. 4 and 5, the skin measuring device 1 is part of a wristwatch 20. The wristwatch 20 has a dial 21 with watch hands 22 which are arranged on the housing base 4 on the opposite side of the housing 2 and thus on a side facing away from the sample stage 10. The skin measuring device 1 also has a USB device connector 19 via which the spectroscopic data gathered by the skin measuring device 1 can be read out by an evaluation unit such as, a smartphone. It is also conceivable for the skin measuring device 1 to have a transmitting unit by which the spectroscopic data gathered by the skin measuring device can be transmitted wirelessly, whereby the data can be wirelessly received by the evaluation unit. The evaluation of the spectroscopic data can take place in the evaluation unit. It is also conceivable for the evaluation unit to have a display device for displaying spectroscopic data and/or the evaluated data.

It is understood that the foregoing description is that of the exemplary embodiments of the invention and that various changes and modifications may be made thereto without departing ton the spirit and scope of the invention as defined in the appended claims.

### LIST OF REFERENCE NUMERALS

1 skin measuring device
2 housing
3 press-on frame
4 housing base
5 window
6 encircling means
7 lug
8 ATR Infrared Spectrometer
9 ATR crystal
10 sample stage
11 entrance face
12 exit face
13 first reflection face
14 second reflection face
15 first infrared light source
16 second inflated tight source
17 exit opening
18 infrared light line detector
19 USB device connector
20 wristwatch
21 dial
22 watch hands

What is claimed is:

1. A skin measuring device for spectroscopically measuring a skin of a body part, the skin measuring device comprising:
a press-on frame having a window and a flat face;
an ATR infrared spectrometer having an ATR crystal, the ATR crystal including a sample stage and being attached to the press-on frame,
wherein the sample stage:
is arranged in the window,
engages with the press-on frame,
faces in the same direction as the flat face of the press-on frame, and
is in contact with the skin to allow a spectroscopic measurement of the skin with the ATR spectrometer; and
an encircling means configured for surrounding the body part and to support the skin measuring device on the body part,
wherein the press-on frame is pressed by the encircling means with the flat face against the skin of the body part when the skin measuring device is worn.

2. The skin measuring device according to claim 1, wherein the body part is an arm of a human being and the encircling means is a wristband.

3. The skin measuring device according to claim 2, wherein the wristband is elastic and is adapted with its length to the body part such that the wristband is elongated when the skin measuring device is worn, and
wherein the press-on frame is pressed in a comfortably flat manner with its flat face against the skin of the body part.

4. The skin measuring device according to claim 1, wherein the ATR crystal is arranged in the window in such a way that the sample stage and the flat face of the press-on frame lie in the same plane.

5. The skin measuring device according to claim 1, further comprising a housing with a housing base, wherein the housing base is the press-on frame.

6. The skin measuring device according to claim 1, wherein the ATR crystal is made of a material having a refractive index n₁ greater than 1.5 in a range of infrared radiation.

7. The skin measuring device according to claim 1, wherein the ATR crystal is made of a material having a refractive index n₁ of from 2.3 to 2.8 in a range of infrared radiation.

8. The skin measuring device according to claim 6, wherein:
the ATR crystal has two reflection faces,
the two reflection faces are inclined relative to the sample stage,
inclination angles of the two reflection faces are selected to cause infrared light radiation to be transmitted at an angle θ relative to the sample stage,
the angle θ is in a range of from arcsine (1.3/n₁)+5° to arcsine (1.3/n₁)+10°, and
the angle θ is formed by the infrared light radiation and a line that is perpendicular to the sample stage.

9. The skin measuring device according to claim 7, wherein:
the ATR crystal has two reflection faces,
the two reflection faces are inclined relative to the sample stage,
inclination angles of the two reflection faces are selected to cause infrared light radiation to be transmitted at an angle θ relative to the sample stage,
the angle θ is in a range of from arcsine (1.3/n₁)+5° to arcsine (1.3/n₁)+10°, and
the angle θ is formed by the infrared light radiation and a line that is perpendicular to the sample stage.

10. The skin measuring device according to claim 1, further comprising:
a device connector configured to provide the spectroscopic data determined by the skin measuring device to be read out, and
a transmitting unit configured to transmit the spectroscopic data determined by the skin measuring device wirelessly.

11. The skin measuring device according to claim 1, fluffier comprising:
a transmitting unit configured to transmit the spectroscopic data determined by the skin measuring device wirelessly.

12. The skin measuring device according to claim 10, further comprising:
an analysis unit configured to:
read out the spectroscopic data via the device connector and receive the spectroscopic data transmitted by the transmitting unit wirelessly, and
evaluate the spectroscopic data.

13. The skin measuring device according to claim 11, further comprising:
an analysis unit configured to:
receive the spectroscopic data transmitted by the transmitting unit wirelessly, and
evaluate the spectroscopic data.

14. A wristwatch with a skin measuring device according to claim 1.

15. The wristwatch according to claim 14, further comprising a dial arranged facing away from the sample stage.
